(19) United States
(12) Patent Application Publication
(10) Pub. No.: US 2026/0256684 A1
(43) Pub. Date: Sep. 3, 2026

(54) Method for Preparing Fiber Deodorant Composition

(71) Applicant: Bioworks, Busan (KR)

(72) Inventor: Jin Won Park, Busan (KR)

(21) Appl. No.: 18/998,335

(22) PCT Filed: Jul. 12, 2023
(86) PCT No.: PCT/KR2023/009913

§ 371 Date: Mar. 10, 2025

(30) Foreign Application Priority Data
Aug. 1, 2022 (KR) 10-2022-0095372

(51) Int. Cl. A61K 8/9789; A61K 8/362; A61K 8/60; A61Q 5/02; A61Q 15/00

(52) U.S. Cl. CPC A61K 8/9789; A61K 8/362; A61K 8/60; A61Q 5/02; A61Q 15/00

(57) ABSTRACT

The present disclosure provides a fiber deodorant composition and a method for preparing the same, the composition comprising 1-5 wt % of a grapefruit extract and 40-60 wt % of palm oil purified glycerin, with the remainder being purified water, and simultaneously exhibiting excellent cleaning effects, sterilizing effects and deodorizing effects while being highly safe for the human body. Therefore, unpleasant odors stuck in various clothes due to sweat or the like can be easily removed without washing fibers, and damage to fiber products can be avoided. Further, the present disclosure provides: a fiber deodorant composition comprising only the single active ingredient bioflavonoid Naringin contained in grapefruit extract which is a composite substance, wherein the stability and safety of the fiber deodorant composition is further improved by using 25-45° C. glycerin in order to improve the water solubility of the Naringin.

### TECHNICAL FIELD

The present disclosure relates to a fiber deodorant composition and a method for preparing the same, and more specifically, to a fiber deodorant composition that, by being formulated to include 1-5 wt % of grapefruit extract, 40-60 wt % of palm oil purified glycerin, with the remainder being purified water, can easily remove unpleasant odors stuck in various clothes due to sweat and the like without having to wash the fiber, and at the same time avoid damaging the fiber products, and a method for preparing the same.

### BACKGROUND

Recently, various deodorants are being used in households or offices to enhance pleasantness in everyday life. Conventional deodorants employ organic chemical components or strong fragrances to dilute unpleasant odors, thereby temporarily eliminating them. Although they provide an immediate deodorizing effect, they have problems because their short duration necessitates frequent reapplication. Not only that, they have the drawback of being ineffective in removing long-standing bacteria, fungi, and viruses.

More specifically, the malodorous substances commonly encountered in daily life include amine compounds such as basic ammonia and trimethylamine, sulfur compounds such as acidic hydrogen sulfide and methyl mercaptan, and aldehyde compounds such as acetaldehyde, which has a low boiling point. These malodorous substances are also classified as basic odors, acidic odors, and volatile odors.

For basic odors, there are nitrogen compounds—such as ammonia and amines—that are generated from sweat smell, body smell, bathroom smell, meat smell or smell from fish dishes. For acidic odors, there are sulfur compounds—such as hydrogen sulfide and mercaptans—that are produced from smells like kimchi or vegetable cooking as well as bacterial and fungal smells. In addition, volatile odors include tobacco smell.

These odor-causing agents, which induce such unpleasant sensations, can permeate fiber products such as clothing or curtains, and carpets, leaving behind undesirable smells that may cause severe discomfort in sensitive individuals. Moreover, these smells can create an environment conducive to the growth of bacteria or fungi on clothing or fiber products, potentially leading to skin inflammation or various skin disorders. Consequently, there is growing interest in households or offices in fiber deodorants that can easily remove smells such as those from sweat embedded in various clothing without the need for washing and at the same time without damaging the fiber products.

In this regard, Korean Laid-open Patent No. 2008-0103172 (hereinafter referred to as Patent Document 1) discloses a deodorant composition for clothing and fiber products comprising: a) a chemical deodorant selected from a group consisting of betaine compounds, organic acids or salts thereof, amines, polyols, and mixtures thereof; and b) an inorganic metal coordination compound containing copper or zinc metal dispersed in an organic acid solution. Moreover, Korean Laid-open Patent No. 2013-0001866 (hereinafter referred to as Patent Document 2) discloses a liquid antibacterial deodorant preparation method that includes the steps of: mixing ceramic powder with a metal catalyst in a liquid component to prepare a ceramic mixed solution; pulverizing the ceramic mixed solution to produce a ceramic nano liquid with particle sizes ranging from 3 nm to 600 nm; and diluting the ceramic nano liquid in an inorganic solvent.

However, Patent Documents 1 and 2 do not disclose a fiber deodorant composition that simultaneously exhibits excellent cleaning, bactericidal, antiviral, and deodorizing actions while being formulated to include grapefruit extract, that is a natural plant extract that is highly safe for the human body, palm oil glycerin, and purified water. In particular, they do not disclose a fiber deodorant composition in which safety and stability are further enhanced by using glycerin of 25° C. to 45° C. to improve the solubility of the bioflavonoid naringin.

### DISCLOSURE OF THE INVENTION

### Technical Problem

A purpose of the present disclosure is to provide a biocompatible fiber deodorant composition that simultaneously exhibits excellent cleaning, bactericidal, antiviral, and deodorizing actions, and that by being formulated to include 1-5 wt % of grapefruit extract, that is a natural plant extract with high safety for the human body, 40-60 wt % of palm oil purified glycerin, with the remainder being purified water.

Furthermore, another purpose of the present disclosure is to provide a fiber deodorant composition that, among the composite components of grapefruit extract, includes only a single active ingredient, in particular, bioflavonoid naringin, and further enhances stability and safety by using glycerin of 25° C. to 45° C. to improve the solubility of the naringin component.

Furthermore, another purpose of the present disclosure is to provide a fiber deodorant composition that uses, in addition to the grapefruit extract, a four-component composite organic acid component as an active ingredient comprising 1-5 wt % of acerola fruit extract vitamin C, 1-5 wt % of acetic acid extracted from apple fermentation liquid, and 1-5 wt % of organic citric acid extracted from tangerine and fruits.

However, the purposes of the present disclosure are not limited to the purposes mentioned above, and other purposes not specifically mentioned will be clearly understood by those skilled in the art from the following description.

### Technical Solution

To achieve the above purposes, the present disclosure provides a fiber deodorant composition and a method for preparing the same, which comprises 1-5 wt % of grapefruit extract, 40-60 wt % of palm oil purified glycerin, and the remainder of purified water.

Furthermore, according to the present disclosure, a fiber deodorant composition and a method for preparing the same are provided, characterized in that the grapefruit extract includes only a single active ingredient, bioflavonoid naringin.

Additionally, according to the present disclosure, a fiber deodorant composition and a method for preparing the same are provided, characterized in that glycerin of 25° C. to 45° C. is used to improve the solubility of bioflavonoid naringin.

Furthermore, according to the present disclosure, a fiber deodorant composition and a method for preparing the same are provided, characterized in that it additionally comprises 1-5 wt % of acerola fruit extract vitamin C, 1-5 wt % of acetic acid extracted from apple fermentation liquid, and 1-5 wt % of organic citric acid extracted from tangerine and fruits.

### Advantageous Effects

According to the present disclosure, the fiber deodorant composition, being formulated to include 1-5 wt % of grapefruit extract, that is a natural plant extract with high safety for the human body, 40-60 wt % of palm oil purified glycerin, with the remainder being purified water, can simultaneously exhibit excellent cleaning, bactericidal, antiviral, and deodorizing actions.

Furthermore, the fiber deodorant composition according to the present disclosure, by employing glycerin of 25° C. to 45° C. and purified water as the solvent and including the bioflavonoid naringin as the single active ingredient within the composite grapefruit extract, can greatly enhance the solubility of the naringin component, thereby further improving stability and safety.

Furthermore, the fiber deodorant composition according to the present disclosure, by including in addition to the grapefruit extract a four-component composite organic acid component as an active ingredient—which further comprises 1-5 wt % of acerola fruit extract vitamin C, 1-5 wt % of acetic acid extracted from apple fermentation liquid, and 1-5 wt % of organic citric acid extracted from tangerine and fruits—features a synergistic effect wherein each component interacts organically to more effectively eliminate unpleasant odors caused by basic compounds such as ammonia and trimethylamine, and additionally has the advantage of reinforcing the deodorizing effect against various malodors caused by acidic compounds such as sulfur compounds, that is, hydrogen sulfide and methyl mercaptan, or aldehyde compounds, that is, acetaldehyde.

### BRIEF DESCRIPTION OF THE DRAWINGS

FIGS. 1 to 4 are drawings showing the change in the dissolution state according to solvents in Examples 1 to 3 and Comparative Examples 1 to 5.

FIG. 5 is a drawing showing the change in reflectance according to the temperature change in Example 1 and Comparative Example 2.

FIG. 6 is a test report showing the deodorizing efficacy of Example 1.

FIG. 7 is a test report showing the deodorizing efficacy of Comparative Example 1.

### DETAILED DESCRIPTION

Prior to describing the present disclosure in detail, it should be understood that the terms used in this specification are employed solely to describe specific embodiments and are not intended to limit the scope of the present disclosure solely by the appended claims. All technical and scientific terms used in this specification have the same meaning generally understood by those of ordinary skill in the art, unless otherwise indicated.

Throughout this specification and the claims, unless otherwise mentioned, the term “comprise, comprises, comprising” is used to mean that the items, steps, or groups of items and steps mentioned are included, and it is not used to exclude any other item, step, or group of items and steps.

Meanwhile, various embodiments of the present disclosure may be combined with any other embodiments, unless there is a clear indication to the contrary. In particular, any feature indicated as preferable or advantageous may be combined with any other feature and features indicated as preferable or advantageous.

Hereinafter, the present disclosure is described in more detail.

A fiber deodorant composition according to one embodiment of the present disclosure is formulated by including 1-5 wt % of grapefruit extract, 40-60 wt % of palm oil purified glycerin, with the remainder being purified water.

The grapefruit extract used in the present disclosure is composed of a complex of components such as tannins, polyphenols, tocopherols, and the bioflavonoid naringin, and these components are characterized by their ability to remove odor substances through chemical deodorizing actions such as neutralization and addition, substitution, reduction of malodor gas components such as ammonia, trimethylamine, hydrogen sulfide, and methyl mercaptan, as well as through physical or encapsulation, adsorption, absorption actions.

The fiber deodorant composition of the present disclosure is preferably one that includes only a single active ingredient, the bioflavonoid naringin, among the complex components of the grapefruit extract. More specifically, the bioflavonoid naringin is a component that is mainly present in the inner peel of well-ripened grapefruit, providing deodorizing effects as well as bacteriostatic action that halts the growth of microorganisms and antibacterial action that sterilizes microorganisms, and has a chemical formula of C₂₇H₃₂O₁₄, a molecular weight of 580.54 g/mol, and a melting point of 166° C.

In the present disclosure, while the bioflavonoid naringin component provides excellent deodorizing and antibacterial properties, it not only exhibits the characteristic of being poorly soluble in certain solvents but also has the drawback that its solubility varies with temperature changes.

In the present disclosure, in order to provide the optimal dissolution state and to offer the effect of maintaining that dissolution state for an extended period by modifying the insoluble characteristic of the bioflavonoid naringin component, it is particularly preferable to use glycerin of 25° C. to 45° C. mixed with purified water as the solvent.

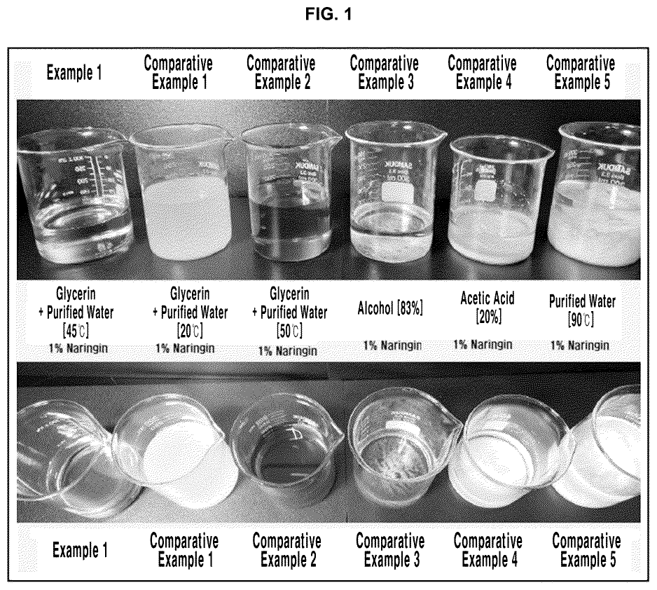

In the present disclosure, when using glycerin of 45° C., as shown in Example 1 of FIG. 1, the naringin component is dissolved transparently without any precipitation being formed, thereby providing the effect of enhancing the stability of the deodorant composition. On the other hand, when using glycerin below 25° C., as shown in Comparative Example 1 of FIG. 1, there is the drawback that the naringin component is not well dissolved, accordingly the deodorizing effect cannot be expected, and due to layer separation, an unpleasant odor is emitted. Furthermore, when using glycerin above 45° C., as shown in Comparative Example 2 of FIG. 1, there is the drawback that browning phenomenon of the bioflavonoid component occurs due to the temperature increase.

In other words, in the present disclosure, the naringin aqueous solution obtained by mixing glycerin of 25° C. to 45° C. with the remainder of purified water, compared to naringin powder, maximizes an unlimited range of application, allows for concentration adjustment and accordingly high stability, and can maximize ease of use. In addition, since separate organic solvents are not required, maintaining purity is easy, and unnecessary processes and equipment for the removal of used organic solvents are not needed at all, thereby simultaneously satisfying both worker protection and user safety. Moreover, according to the present disclosure, the preparation method can not only be simplified but also increase versatility by enabling the mass production of water-soluble substances through a process that does not use any organic solvents or surfactants.

Meanwhile, in the present disclosure, the palm oil purified glycerin serves to remove basic and volatile odor substances and, in particular, provides the effect of exhibiting excellent antibacterial effect through the inhibition of bacteria and fungi, and also performs deodorizing action through chemical reactions—such as neutralization or addition, condensation—with the basic and volatile functional groups of various odor components by means of its highly reactive terminal hydroxyl groups.

In the present disclosure, the palm oil purified glycerin is used within a range of 40-60 wt %; if used below 40 wt %, the binding force with the bioflavonoid naringin component is weakened, resulting in the separation of the bioflavonoid naringin component, and if used in excess of 60 wt %, the product viscosity increases, rendering it unsuitable for application in spray-type containers-which are highly convenient for fiber deodorant products.

In the present disclosure, in addition to the grapefruit extract, by employing as the active ingredient a four-component composite organic acid component comprising 1-5 wt % of acerola fruit extract vitamin C, 1-5 wt % of acetic acid extracted from apple fermentation liquid, and 1-5 wt % of organic citric acid extracted from tangerine and fruits, each component interacts organically to not only exhibit a synergistic effect that more effectively removes the unpleasant odor caused by basic compounds such as ammonia and trimethylamine, but also has the advantage of reinforcing the deodorizing effect against various malodors caused by acidic compounds such as hydrogen sulfide and methyl mercaptan or aldehyde compounds such as acetaldehyde.

Meanwhile, a feature of the fiber deodorant composition of the present disclosure is that it does not cause fiber damage such as discoloration or staining, fading on various natural fibers such as cotton or synthetic fibers such as polyester, and even when exposed to sunlight, it does not cause fiber damage such as deformation of the fiber shape, or color changes and staining, thereby having the advantage of being safely used on textile products such as clothing, carpets, and curtains.

In addition, in the fiber deodorant composition according to the present disclosure, a fragrance that emits a fresh and subtle aroma may be further included to impart a pleasant scent to the fiber product after deodorization. Such a fragrance can be used in an extremely small amount, within a range that does not affect the physical stability or deodorizing effect of the composition of the present disclosure.

Hereinafter, the present disclosure will be described in more detail with reference to embodiments. Moreover, the scope of the present disclosure is not limited to the following embodiments.

### EXAMPLE 1

By mixing 1 wt % of the bioflavonoid naringin component as grapefruit extract, 50 wt % of 45° C. palm oil purified glycerin, and the remainder of purified water, the fiber deodorant composition of the present disclosure was obtained.

### EXAMPLE 2

Except for using 35° C. palm oil purified glycerin instead of 45° C. palm oil purified glycerin, the process was carried out in the same manner as in Example 1 to obtain the fiber deodorant composition.

### EXAMPLE 3

Except for using 25° C. palm oil purified glycerin instead of 45° C. palm oil purified glycerin, the process was carried out in the same manner as in Example 1 to obtain the fiber deodorant composition.

### COMPARATIVE EXAMPLE 1

Except for using 20° C. palm oil purified glycerin instead of 45° C. palm oil purified glycerin, the process was carried out in the same manner as in Example 1 to obtain the fiber deodorant composition.

### COMPARATIVE EXAMPLE 2

Except for using 50° C. palm oil purified glycerin instead of 45° C. palm oil purified glycerin, the process was carried out in the same manner as in Example 1 to obtain the fiber deodorant composition.

### COMPARATIVE EXAMPLE 3

Except for using 83 wt % alcohol instead of 45° C. palm oil purified glycerin, the process was carried out in the same manner as in Example 1 to obtain the fiber deodorant composition.

### COMPARATIVE EXAMPLE 4

Except for using 20 wt % acetic acid instead of 45° C. palm oil purified glycerin, the process was carried out in the same manner as in Example 1 to obtain the fiber deodorant composition.

### COMPARATIVE EXAMPLE 5

Without using 45° C. palm oil purified glycerin, by mixing 1 wt % of the bioflavonoid naringin component as grapefruit extract with the remainder of purified water of 90° C., the fiber deodorant composition was obtained.

### TEST EXAMPLE 1

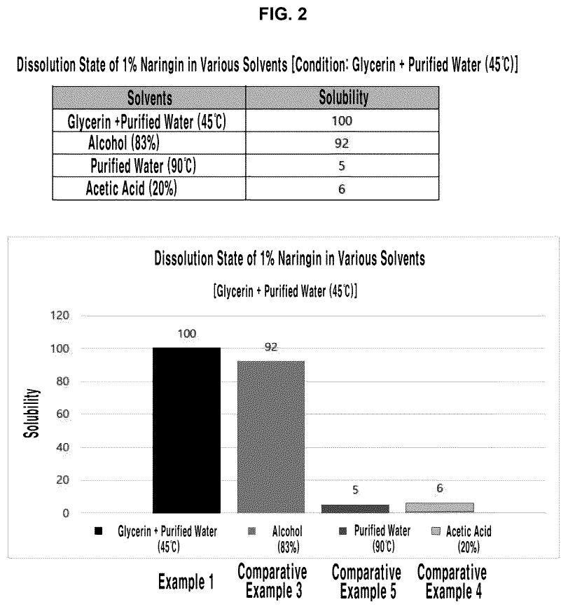

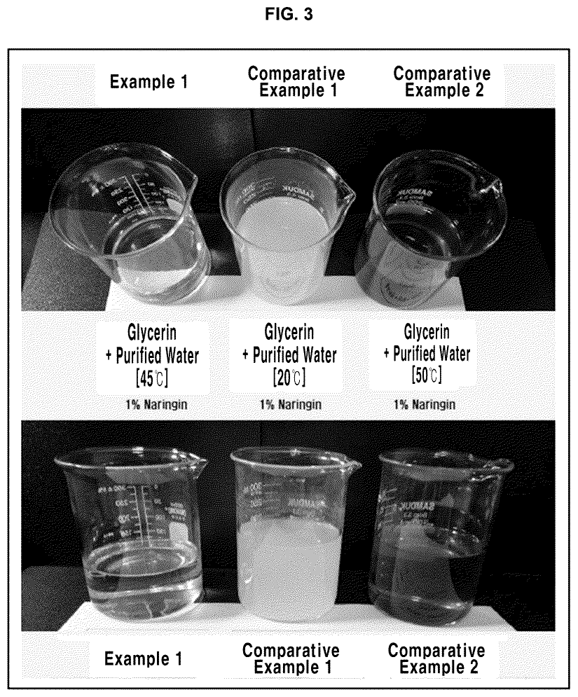

The fiber deodorant composition obtained in Example 1 and Comparative Examples 1 to 5 were placed in a beaker and left for 48 hours, after which the dissolution state of the compositions was checked. The results of this check are shown in FIGS. 1 to 3.

As shown in FIGS. 1 to 3, in the case of Example 1, in which 1 wt % of the naringin component was dissolved in a solvent composed of 45° C. glycerin and purified water, it was found that no precipitation was observed and that it was well dissolved transparently without any change in color.

In contrast, in the case of Comparative Example 1, where the temperature of the glycerin and purified water was changed to 20° C., the solution became turbid with precipitation observed, and in the case of Comparative Example 2, where the temperature of the glycerin and purified water was changed to 50° C., it was found that the solution turned a very dark brown, confirming that it is in a state impossible for use in fiber products. In particular, when using the composition of Comparative Example 2 as a fiber deodorant, immediate washing is required, and if used repeatedly, a situation may arise in which it must be discarded; thus, it can be seen that it must never be used on light-colored fiber products.

Meanwhile, in the case of Comparative Example 3, where alcohol 83 wt % was used as the solvent, due to its low reactivity, precipitation occurred as a result of unreacted material, and not only did the color of the combined solution fail to remain clear, but it was also found that, as the volatile component alcohol evaporated into the air over time, it tended to solidify again. In other words, while the composition of Example 1 maintained an overall continuously transparent state with excellent stability as a fiber deodorant, the composition of Comparative Example 3 not only exhibited precipitation but also underwent discoloration or solidification over time, thereby confirming that when sprayed onto fiber products or clothing, there is a concern that the surface of the fibers may be altered or damaged after some time, resulting in significantly inferior stability as a fiber deodorant.

In addition, it was confirmed that the use of organic solvents has the disadvantage that, when used on animal fiber products woven from protein components whose basic ingredient is amino acids (see Table 1 below: Types of Animal Fibers), it may cause negative or critically detrimental defects in the fiber material or management issues.

TABLE 1
Classification of Animal Fibers

| Classification | | | Type |
|---|---|---|---|
| Animal | General Fibers | | Wool(Sheep) |
| Hair | Special | Camelid | Alpaca, Huarizo, Vincuna, Llama |
| Fibers | Fibers | Fibers | |
| | | Goat Fibers | Cashmere, Mohair, Qiviut, Cashgora |
| | | Fur Fibers | Beaver, Fox, Mink, Chinchilla, Rabbit |
| | | Other Fibers | Horse Hair, Cow Hair |

| | |
|---|---|
| Extruded Fibers | Silk |

| | |
|---|---|
| Regenerated Protein Fibers | Animal Hair, All Protein Fibers |
| Animal Protein Fibers | Milk Protein, Chinon |

Furthermore, in the case of Comparative Example 4, which uses 20 wt % acetic acid, due to its low solubility, a large amount of unreacted precipitate is produced, making it difficult to expect a deodorizing effect, and moreover, if used, the inherent acetic acid odor causes very severe unpleasantness; in the case of Comparative Example 5, which uses only 90° C. purified water, it was found that there is almost no solubility, with most of the material not dissolving, which is a drawback that renders it uncommercializable.

### TEST EXAMPLE 2

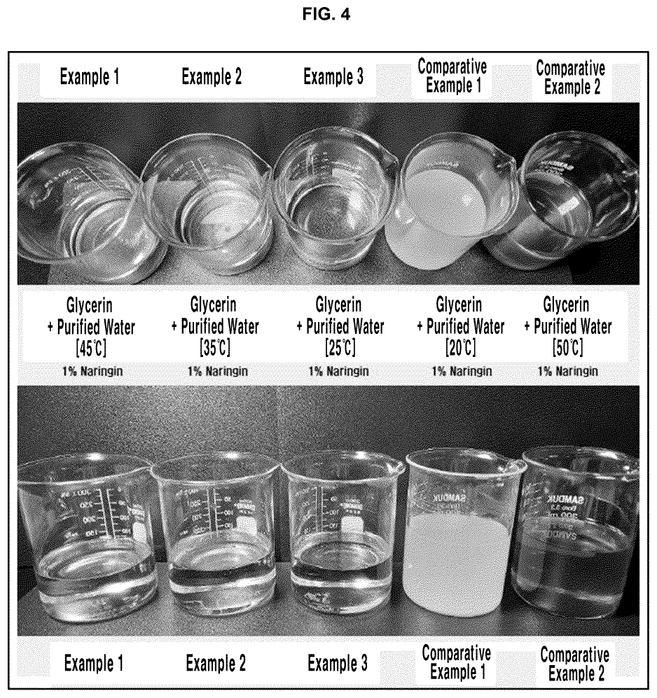

The fiber deodorant compositions obtained in Examples 1 to 3 and Comparative Examples 1 to 2 were placed in a beaker and left for 48 hours, after which the dissolution state of the compositions was examined. The results of this examination are shown in FIG. 4.

As shown in FIG. 4, in the case of Examples 1 to 3, in which 1 wt % of the naringin component was dissolved in a solvent composed of glycerin and purified water of 45° C., 35° C., and 25° C. respectively, no precipitation was observed and it was found to be well dissolved transparently without any change in color; whereas in the case of Comparative Examples 1 and 2, where the temperature was changed to 20° C. and 50° C. respectively, the solution became turbid with precipitation observed or turned very dark brown. From these results, it can be confirmed that the effect of the present disclosure can be obtained only when the temperature range of 25° C. to 45° C. is satisfied, whereas if the temperature range is exceeded, the effect of the present disclosure cannot be obtained.

### TEST EXAMPLE 3

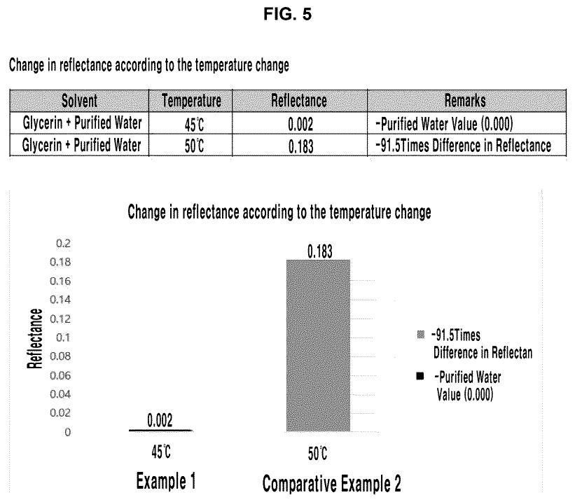

The change in reflectance according to temperature variation of the fiber deodorant compositions obtained in Example 1 and Comparative Example 2 was measured, and the results are shown in FIG. 5.

As shown in FIG. 5, while in the case of Example 1 the reflectance was found to be very low, in the case of Comparative Example 2 a reflectance difference of approximately 91.5 times compared to Example 1 was observed. This means that if used as a fiber deodorant, it may leave traces on the fiber immediately upon use; therefore, the composition of Comparative Example 2 is unsuitable as a fiber deodorant, whereas the composition of Example 1 can be confirmed to be very suitable for use as a fiber deodorant.

### TEST EXAMPLE 4

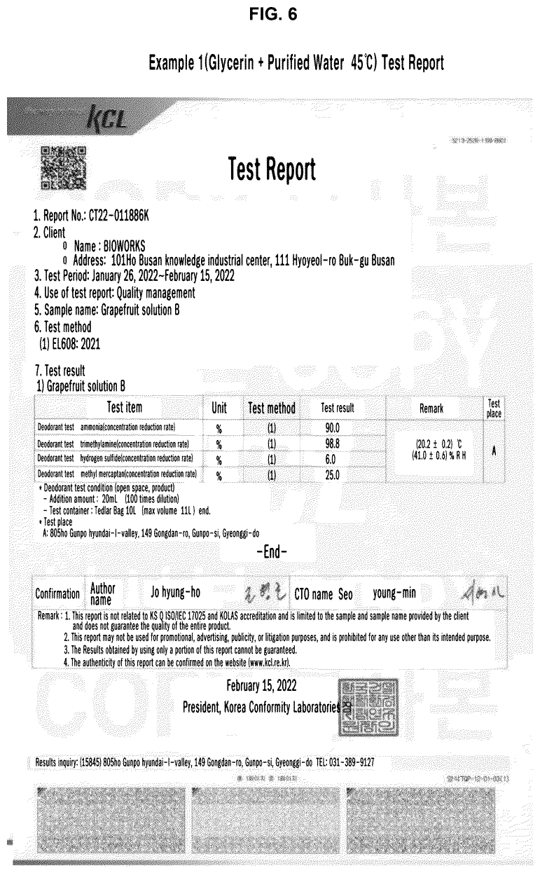

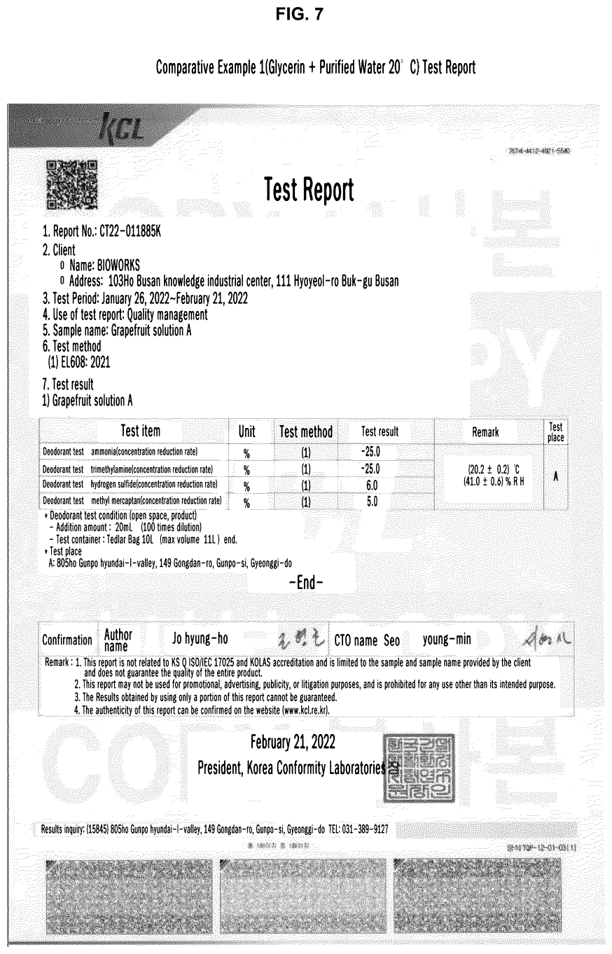

The deodorizing effect of the fiber deodorant compositions obtained in Example 1 and Comparative Example 1 was tested, and the results are shown in FIGS. 6 to 7.

As shown in FIG. 6, in the case of Example 1, a very excellent effect was provided with 90% removal of ammonia, 98% removal of trimethylamine, 6% removal of hydrogen sulfide, and 25% removal of methyl mercaptan; whereas in the case of Comparative Example 1, it was observed that due to layer separation an unpleasant odor was emitted, and particularly as shown in FIG. 7, not only were negative test results obtained for ammonia and trimethylamine, but only 6% of hydrogen sulfide and 5% of methyl mercaptan were removed, indicating an overall inferior effect, from which it can be confirmed that no excellent deodorizing effect can be expected.

The scope of the present disclosure is not limited to the above-described embodiments, but may be implemented in various forms of embodiments within the scope of the appended claims. It is deemed to be within the scope of the claims of the present disclosure to the extent that anyone skilled in the art can make modifications without departing from the gist of the present disclosure as claimed in the claims.

What is claimed is:

1. A method for preparing a fiber deodorant composition, comprising mixing 1-5 wt % of grapefruit extract, 40-60 wt % of palm oil purified glycerin, and the remainder of purified water.

2. The method according to claim 1,
wherein the grapefruit extract comprises only a single active ingredient, bioflavonoid naringin.

3. The method according to claim 2,
wherein glycerin of 25° C. to 45° C. is used to improve the solubility of the bioflavonoid naringin.

4. The method according to claim 1,
further comprising 1-5 wt % of acerola fruit extract vitamin C, 1-5 wt % of acetic acid extracted from apple fermentation liquid, and 1-5 wt % of organic citric acid extracted from tangerine and fruits.
